(19) United States
(12) Patent Application Publication
(10) Pub. No.: US 2026/0110701 A1
(43) Pub. Date: Apr. 23, 2026

(54) METHODS OF DETECTING MICROPLASTICS IN BIOLOGICAL SAMPLES

(71) Applicant: CONTIGUOUS LABS, INC., DOVER, DE (US)

(72) Inventors: Jolieke Van Oosterwijk, Dover, DE (US); Reese Purnell Hodges IV, Dover, DE (US)

(21) Appl. No.: 19/487,630

(22) PCT Filed: May 24, 2024
(86) PCT No.: PCT/US2024/031000

§ 371 Date: Nov. 25, 2025

Related U.S. Application Data
(60) Provisional application No. 63/469,345, filed on May 26, 2023.

(51) Int. Cl. G01N 33/84; G01N 33/53

(52) U.S. Cl. CPC G01N 33/84; G01N 33/5308; G01N 2430/60

(57) ABSTRACT

The present disclosure relates generally to a method for identifying at least one microplastic in a protein-containing liquid biological sample, wherein the method comprises: heating the protein-containing liquid biological sample to provide a protein-containing sediment and a liquid phase disposed above the sediment; separating the liquid phase from the protein-containing sediment; evaporating the liquid phase to provide an analyte; treating the analyte with a depolymerization solution comprising an organic phase and an aqueous phase; extracting the analyte from the depolymerization solution in the organic phase of the depolymerization solution; filtering the organic phase to provide an analyte residue as the oversize; and identifying the at least one microplastic in the analyte residue with size-exclusion liquid chromatography and mass spectrometry.

### CROSS-REFERENCE TO RELATED APPLICATIONS

This application claims the benefit of U.S. Provisional Patent Application No. 63/469,345, filed May 26, 2023, which is hereby incorporated herein by reference in its entirety.

### 1. FIELD

The present disclosure relates generally to methods of identifying microplastics in biological samples.

### 2. TECHNICAL BACKGROUND

Plastics are ubiquitous to everyday life and as the use of plastics has increased, efforts to understand the effects of such materials on both environmental and biological systems have increased. In particular, focus has turned to how microplastics infiltrate the human body and impact human health. However, in order to understand the effects microplastics have on human health, detection of such materials is first required.

Currently, there are few detection methods available that can identify microplastics in common biological samples, like blood or urine. Of the methods currently used, however, these methods are often complicated, requiring multiple sample treatment steps and chemical modification of the microplastics. Such methods are time consuming and costly. Accordingly, there is a need in the art to provide an easier and simpler method for detecting microplastics in biological samples.

### SUMMARY

Currently disclosed are methods for identifying microplastics in biological samples. In particular, the present disclosure provides methods for identifying microplastics in blood and urine samples. The presently disclosed methods involve extracting microplastics from a biological sample and identifying the microplastics.

Accordingly, in one aspect, the present disclosure provides a method for identifying at least one microplastic in a protein-containing liquid biological sample, wherein the method comprises:
heating the protein-containing liquid biological sample to provide a protein-containing sediment and a liquid phase disposed above the sediment;
separating the liquid phase from the protein-containing sediment;
evaporating the liquid phase to provide an analyte;
treating the analyte with a depolymerization solution comprising an organic phase and an aqueous phase;
extracting the analyte from the depolymerization solution in the organic phase of the depolymerization solution;
filtering the organic phase to provide an analyte residue as the oversize; and
identifying the at least one microplastic in the analyte residue with size-exclusion liquid chromatography and mass spectrometry.

In another aspect, the present disclosure provides a method for identifying at least one microplastic in a liquid blood sample, wherein the method comprises:
heating the liquid blood sample to provide a protein-containing sediment and a liquid phase disposed above the sediment,
separating the liquid phase from the protein-containing sediment;
evaporating the liquid phase to provide an analyte;
treating the analyte with a depolymerization solution comprising an organic phase and an aqueous phase;
extracting the analyte from the depolymerization solution in the organic phase of the depolymerization solution;
filtering the organic phase to provide an analyte residue as the oversize; and
identifying the at least one microplastic in the analyte residue with size-exclusion chromatography and mass spectrometry.

In another aspect, the present disclosure provides a method for identifying at least one microplastic in a non-protein-containing liquid biological sample, wherein the method comprises:
providing a non-protein-containing liquid biological sample;
evaporating the non-protein-containing liquid biological sample to provide an analyte;
treating the analyte with a depolymerization solution comprising an organic phase and an aqueous phase;
extracting the analyte from the depolymerization solution in the organic phase of the depolymerization solution;
filtering the organic phase to provide an analyte residue as the oversize; and
identifying the at least one microplastic in the analyte residue.

### BRIEF DESCRIPTION OF FIGURES

The accompanying drawings are included to provide a further understanding of the methods of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIG. 1A is a liquid chromatograph and mass spectrum of a polypropylene standard, as measured using an Enhanced Multi-Charge (EMC) ionization method.

FIG. 1B is a liquid chromatograph and mass spectrum of a polypropylene standard, as measured using an Enhanced Product Ion (EPI) ionization method.

FIG. 2A is a liquid chromatograph and mass spectrum of a polyethylene terephthalate standard, as measured using an Enhanced Multi-Charge (EMC) ionization method.

FIG. 2B is a liquid chromatograph and mass spectrum of a polyethylene terephthalate standard, as measured using an Enhanced Product Ion (EPI) ionization method.

FIG. 3A is a liquid chromatographs and mass spectrum of a poly(methyl methacrylate) standard, as measured using an Enhanced Multi-Charge (EMC) ionization method.

FIG. 3B is a liquid chromatographs and mass spectrum of a poly(methyl methacrylate) standard, as measured using an Enhanced Product Ion (EPI) ionization method.

FIG. 4A is a liquid chromatograph and mass spectrum of a clear polyethylene standard, as measured using an Enhanced Multi-Charge (EMC) ionization method.

FIG. 4B is a liquid chromatograph and mass spectrum of a clear polyethylene standard, as measured using an Enhanced Product Ion (EPI) ionization method.

FIG. 5A is a liquid chromatograph and mass spectrum of a blood sample, as measured using an Enhanced Multi-Charge (EMC) ionization method.

FIG. 5B is a liquid chromatograph and mass spectrum of a blood sample, as measured using an Enhanced Product Ion (EPI) ionization method.

FIG. 6A is a liquid chromatograph and mass spectrum of a blood sample, as measured using an Enhanced Multi-Charge (EMC) ionization method.

FIG. 6B is a liquid chromatograph and mass spectrum of a blood sample, as measured using an Enhanced Product Ion (EPI) ionization method.

### DETAILED DESCRIPTION

The present disclosure is concerned with methods of identifying microplastics in biological samples. Understanding the effects of microplastics on human health are limited by the ability to accurately and efficiently detect such plastics in biological samples. Currently, methods of identifying microplastics from biological samples are often complicated, requiring multiple sample treatment steps with multiple reagents. Overly complicated methods increase the time, effort, and cost required to test samples, and can often lead to other deleterious outcomes (e.g., unsubstantiated results). Accordingly, the present disclosure focuses on providing methods of identifying microplastics that require the least amount of sample manipulation as possible while maintaining high throughput and accuracy.

In an example embodiment, a method of removing any unwanted substances from the biological sample is disclosed, including extracting the microplastic from the sample, and then identifying the microplastic. Removal of unwanted substances and impurities provides a sample with less impurities that may influence the measurement and identification of the microplastic. For example, when the biological sample contains protein (e.g., as in blood samples), the proteins present are first removed before extracting the microplastic from the sample. By removing the protein for the biological sample, they will not interfere with the collection and identification of the microplastic. Conversely, when the biological sample does not contain proteins (e.g., as in urine samples), no removal step is required.

In an example embodiment, to extract the microplastics, the present disclosure provides the use of a depolymerization solution that includes an organic phase and an aqueous phase that can easily collect the microplastics into the organic phase. Additionally, the use of the depolymerization solution as described herein helps to break up the polymers that make up the microplastics into its monomers, dimers, trimers, or higher order polymers. Without being bound by theory, the present disclosure hypothesizes that the solution disrupts the covalent bonds within the polymer chains to provide polymers with a distribution of sizes. Accordingly, the depolymerization solution provides not only an easy way to extract the microplastics from the biological samples, but also breaks down the microplastics for monomer and/or polymer identification. As used herein, monomer refers to the small molecule that makes up the repeating unit of a polymer. For example, the monomer of polyethylene glycol (PEG) is ethylene glycol. As used herein, polymer refers to a molecule having any number (i.e., n) of repeating monomers. For example, a polymer of polyethylene glycol has an n of at least 2 ethylene glycols.

In an example embodiment, once extracted, the microplastics are then identified, and may be identified in a number of ways. In an example embodiment, the present disclosure describes that it is particularly useful to identify the microplastics with size-exclusion chromatography in combination with mass spectrometry. Using size-exclusion chromatography provides separation of the extracted monomers based on size while using mass spectrometry provides an accurate measurement of the mass of each separated monomer. The mass measured is a unique qualifier of the monomer that makes up the microplastic polymer. As such, the present disclosure improves the identification of the microplastics from the biological sample based on both size and weight, providing two factors to ultimately identify the microplastics in biological samples. In doing so, the present disclosure provides an improved and more accurate method for identifying microplastics in biological samples.

As such, in one aspect, the present disclosure as described herein provides a method for identifying at least one microplastic in a protein-containing liquid biological sample, wherein the method includes heating the protein-containing liquid biological sample to provide a protein-containing sediment and a liquid phase disposed above the sediment; separating the liquid phase from the protein-containing sediment; evaporating the liquid phase to provide an analyte; treating the analyte with a depolymerization solution comprising an organic phase and an aqueous phase; extracting the analyte from the depolymerization solution in the organic phase of the depolymerization solution; filtering the organic phase to provide an analyte residue as the oversize; and identifying the at least one microplastics in the analyte residue with size exclusion liquid chromatography and mass spectrometry.

The protein-containing liquid biological sample may be obtained from either a human or an animal. In some embodiments as described herein, the protein-containing liquid biological sample is obtained from a human. In some embodiments as described herein, the protein-containing liquid biological sample is obtained from an animal. The type of animal is not particularly limited. For example, the animal may be selected from a dog, a cat, a mouse or rat, or a bird. As described above, in some embodiments of the disclosure, the liquid biological sample contains protein. For example, in some embodiments, the protein-containing liquid biological sample is a blood sample. In some embodiments as described herein, the blood sample is a liquid whole blood sample. As would be understood by the person of ordinary skill in the art, a liquid whole blood sample comprises red blood cells, white blood cells, platelets, and plasma. In some embodiments as described herein, the liquid whole blood sample is selected from human blood or animal blood (e.g., canine blood, feline blood, racine blood, or avian blood). Additionally, the liquid biological sample may be further treated before the heating step as described herein. For example, the liquid biological sample may be diluted with an aqueous solution. The aqueous solution is not particularly limited. For example, in some embodiments, the aqueous solution may be an aqueous alcohol solution (e.g., at least 10%, at least 25%, at least 50%, or at least 75% aqueous alcohol solution). The alcohol may be selected from methanol, ethanol, or butanol.

As described above, when the biological sample is a protein-containing liquid biological sample, removal of unwanted substances from the sample can advantageously lower any background or interference in the measurement and identification of the microplastic. As such, in some embodiments, the method includes heating the protein-containing liquid biological sample to provide a protein-containing sediment and a liquid phase disposed above the sediment. As would be understood by the person of ordinary skill in the art, heating the protein-containing liquid biological sample can disrupt cellular structures and denature proteins that may be present in the biological sample. In some embodiments as described herein, heating the protein-containing liquid biological sample is conducted at a temperature in the range of 90° C. to 130° C. For example, in various embodiments, heating the protein-containing liquid biological sample is conducted at a temperature in the range of 100° C. to 130° C., or 110° C. to 130° C., or 90 to 120° C., or 100° C. to 120° C., or 110° C. to 120° C. In some embodiments as described herein, heating the protein-containing liquid biological sample is conducted for a time in the range of 5 minutes to 1 hour. For example, in various embodiments, heating the protein-containing liquid biological sample is conducted for a time in the range of 5 to 50 minutes, or 5 to 40 minutes, or 10 minutes to 1 hour, or 10 to 50 minutes, or 10 to 40 minutes, or 20 minutes to 1 hour, or 20 to 50 minutes, or 20 to 40 minutes.

The method of heating the biological sample is not particularly limited. For example, heating the protein-containing liquid biological sample may occur with a water bath, with an oil bath, with a hot plate, with a thermal gun, or in an oven. In various embodiments as described herein, heating the protein-containing liquid biological sample is conducted in a closed system, an open system, or a combination of both. For example, heating the protein-containing liquid biological sample may be conducted in a container comprising a lid and the heating may occur with the lid on (e.g., a closed system) or with the lid off (e.g., an open system). In some embodiments, heating may be conducted with the lid on for a desired amount of time and with the lid off for a desired amount of time.

Heating the protein-containing liquid biological sample provides a protein-containing sediment and a liquid phase disposed above the sediment. The protein-containing sediment may include other materials besides proteins. For example, in some embodiments as described herein, the protein-containing sediment comprises at least one of salts, lipids (e.g., phospholipids or sphingolipids), cholesterol, sugars, or fats. The heating provides a simple and efficient method of separating out proteins from the protein-containing liquid biological sample. This separation ensures that the proteins cannot interfere in the measurement and identification of the microplastics. As such, the background of the measurement of the microplastic is lowered and provides better signal-to-noise to the measurement. Reduced signal-to-noise provides more accurate measurement and identification of the microplastics. Furthermore, by separating out the proteins from the protein-containing liquid biological sample through heating, additional chemical reagents are not necessary. By not including additional reagents, such reagents cannot interfere with subsequent measurement of the samples and do not add any background signal to the measurement.

As described above, heating the biological sample provides a protein-containing sediment and a liquid phase disposed above the sediment. In some embodiments as described herein, the liquid phase comprises microplastics. Microplastics are plastic particles that can be as large as 5 mm. As used herein, microplastics have a particle size (e.g., an average particle size) that is less than 5 mm. Additionally, as used herein, the term “microplastic” encompasses other small plastic particles known in the art (e.g., nanoplastics). In various embodiments as described herein, the liquid phase comprises microplastics with a d₅₀ particle size of no more than 1 mm. As used herein, the d₅₀ particle size is the median particle size, i.e., the size of the particle at which 50% of the particles are of larger particle size and 50% are of smaller particle size. As used herein, “particle size” is the largest dimension of the particle. For example, in various embodiments as described herein, the liquid phase comprises microplastics with a d₅₀ particles size of no more than 500 μm, or no more than 100 μm, or no more than 50 μm, or no more than 20 μm, or no more than 10 μm. In some embodiments as described herein, the liquid phase comprises microplastics with a d₅₀ particle size in the range of 1 μm to 20 μm. For example, in various embodiments, the liquid phase comprises microplastics with a d₅₀ particle size in the range of 1 μm to 15 μm, or 1 μm to 10 μm, or 1 μm to 5 μm, or 2 μm to 20 μm, or 2 μm to 15 μm, or 2 μm to 10 μm, or 2 μm to 5 μm, or 5 μm to 20 μm, or 5 μm to 15 μm, or 5 μm to 10 μm.

Microplastics are derived from larger plastic material. The type (e.g., polymer composition) of the microplastic is not particularly limited and may originate from any plastic material (e.g., a thermoplastic or thermosetting plastic). In some embodiments, the microplastics are thermoplastics. For example, in some embodiments of the present disclosure as described herein, the liquid phase comprises microplastics selected from poly methyl methacrylate (PMMA), polypropylene (PP), polystyrene (PS), polyethylene (PE), polyethylene terephthalate (PET), or mixtures thereof. The liquid phase may comprise other materials. For example, in some embodiments, the liquid phase may comprise at least one of salts, lipids (e.g., phospholipids or sphingolipids), cholesterol, sugars, or fats.

As described above, the method includes separating the liquid phase from the protein-containing sediment. This separation allows for further processing of the liquid phase without contamination from the substances present in the protein-containing sediment as described above. The method of separating the liquid phase from the protein-containing sediment is not particularly limited and may be accomplished by any solid-liquid separation method. As described above, the liquid phase is disposed above the protein-containing sediment. As such, in some embodiments, separating the liquid phase from the protein-containing sediment may include collecting the liquid phase (e.g., by decanting) from above the sediment.

The method as described herein includes evaporating the liquid phase to provide an analyte. The evaporation is not particularly limited and may be accomplished by any evaporation method. The evaporation may be conducted at a temperature and for a time that is sufficient to evaporate the liquid phase to provide an analyte. The temperature at which evaporation occurs depends on the boiling point of the liquid that makes up the liquid phase. As such, the temperature and time for evaporating the liquid phase is not particularly limited. For example, in various embodiments of the disclosure as described herein, evaporating the liquid phase is conducted at a temperature in the range of 90° C. to 130° C., or 100° C. to 130° C., or 110° C. to 130° C., or 90 to 120° C., or 100° C. to 120° C., or 110° C. to 120° C. In various embodiments as described herein, evaporating the liquid phase is conducted for a time in the range of 5 minutes to 1 hour, 5 to 50 minutes, or 5 to 40 minutes, or 10 minutes to 1 hour, or 10 to 50 minutes, or 10 to 40 minutes, or 20 minutes to 1 hour, or 20 to 50 minutes, or 20 to 40 minutes. Evaporating the liquid phase provides an analyte. In some embodiments, the analyte is a solid. In some embodiments of the disclosure as described herein, the analyte comprises microplastics (e.g., as described herein). In some embodiments, the analyte further comprises other materials (e.g., salts, lipids, cholesterol, sugars, fats) not evaporated.

Through evaporation of the liquid phase, the analyte (e.g., comprising microplastics or other materials) is isolated and may be further treated. As such, the method includes treating the analyte with a depolymerization solution comprising an organic phase and an aqueous phase. In some embodiments of the disclosure as described herein, the organic phase is an alcohol. For example, the organic phase may be selected from methanol, ethanol, propanol (e.g., isopropanol), butanol (e.g., tertbutanol or isobutanol), or pentanol. In some embodiments of the disclosure as described herein, the aqueous phase is basic. For example, the aqueous phase may have a pH of greater than 7 (e.g., greater than 8, or greater than 9, or greater than 10, or greater than 11). In some embodiments, the aqueous phase comprises a base selected from sodium hydroxide, potassium hydroxide, sodium carbonate, potassium carbonate, or calcium carbonate. In some embodiments of the disclosure as described herein, the depolymerization solution comprises the organic phase and the aqueous phase in a volume ratio of at least 4:1, or at least 6:1, or at least 8:1, or at least 10:1. In various embodiments as described herein, the depolymerization solution comprises an organic phase and an aqueous phase in a volume ratio in the range of 4:1 to 20:1, or 4:1 to 18:1, or 4:1 to 15:1, or 4:1 to 12:1, or 6:1 to 20:1, or 6:1 to 18:1, or 6:1 to 15:1, or 6:1 to 12:1. In some embodiments, the analyte comprises microplastics. When the analyte comprises microplastics, it is hypothesized, without being bound by theory, that the solution disrupts the covalent bonds within the polymer chains that make up the plastic material to provide monomers, dimers, trimers, or higher order polymers of the plastic material. As such, the depolymerization solution contributes to breaking down the microplastics, allowing for easier identification.

The method includes extracting the analyte from the depolymerization solution in the organic phase of the depolymerization solution. As such, in various embodiments, the organic phase comprises the analyte. In some embodiments of the disclosure as described herein, extracting the analyte from the depolymerization solution comprises heating the depolymerization solution, shaking the depolymerization solution, and collecting the organic phase. The time and temperature at which the depolymerization solution is heated is not particularly limited. For example, in various embodiments as described herein, heating the depolymerization solution is conducted for a time in the range of 10 to 120 minutes, or 10 to 100 minutes, or 10 to 80 minutes, or 10 to 60 minutes, or 20 to 120 minutes, or 20 to 100 minutes, or 20 to 80 minutes, or 20 to 60 minutes. In various embodiments as described herein, heating the depolymerization solution is conducted at a temperature in the range of 50-200° C. (e.g., in the range of 50-175° C., or 50-150° C., or 65-200° C., or 65-175° C., or 65-150° C., or 75-200° C., or 75-175° C., or 75-150° C., or 85-200° C., or 85-175° C., or 85-150° C., or 95-200° C., or 95-175° C., or 95-150° C.). In some embodiments, collecting the organic phase comprises separating the organic phase from the aqueous phase of the depolymerization solution. The separation is not particularly limited and may be accomplished by any liquid-liquid separation. For example, in some embodiments, collecting the organic phase comprises separating by centrifugation the organic phase from the aqueous phase of the depolymerization solution.

In some embodiments as described herein, the method further includes adjusting the pH of the organic phase. In some embodiments, the pH of the organic phase is adjusted to a pH in the range of 2 to 5. For example, in various embodiments, the method further includes adjusting the pH of the organic phase to a pH in the range of 2 to 4 or 2 to 3. The method of adjusting the pH of the organic phase is not particularly limited and may be accomplished by any means known in the art. For example, acid or base may be added to the organic phase to provide the desired pH.

To recover the analyte, the method also includes filtering the organic phase to provide an analyte residue as the oversize. In various embodiments as described herein, filtering the organic phase comprises a filter of at least 200 nm, or 300 nm, or 400 nm, or 500 nm, or 600 nm, or 700 nm. For example, in various embodiments, filtering comprises a filter with a pore size in the range of 100 to 1000 nm, or 100 to 800 nm, or 200 to 1000 nm, or 200 to 800 nm, or 500 to 1000 nm, or 500 to 800 nm. In some embodiments, the analyte residue (i.e., oversize) is provided on the filter. In some embodiments of the disclosure as described herein, the oversize comprises the analyte (e.g., microplastics or other substances) as described herein.

In some embodiments as described herein, the method further comprises preparing the analyte residue for identification. In some embodiments, preparing the analyte residue for identification includes eluting the analyte residue from the filter with an organic solvent, evaporating the organic solvent, and suspending the analyte residue in a carrier solution. In various embodiments as described herein, the organic solvent is selected from methanol, ethanol, pentanol, or butanol. Evaporating the organic solvent may be accomplished by any evaporation method (e.g., as described herein) and is not particularly limited. In some embodiments, the carrier solution comprises acetonitrile and water. For example, in various embodiments, the carrier solution comprises acetonitrile and water in a volume ratio of at least 0.25:1, or 0.5:1, or 0.75:1. In some embodiments, the carrier solution comprises acetonitrile and water in a volume ratio in the range of 0:25 to 1 to 2:1 (e.g., 0.5:1 to 2:1, or 0.75:1 to 2:1, or 0.25:1 to 1.5:1, or 0.5:1 to 1.5:1, or 0.75:1 to 1.5:1). In some embodiments, the carrier solution comprises acetonitrile and water in a volume ratio of 1:1.

As described above, the method as described herein includes identifying the at least one microplastic in the analyte residue. As used herein, the term “identifying” encompasses detecting the presence of the at least one microplastic in the oversize and/or identifying the type (e.g., polymer composition) of the at least one microplastics. In some embodiments, identifying the at least one microplastic in the analyte residue comprises measuring the carrier solution comprising the eluted analyte residue.

In some embodiments as described herein, identifying the at least one microplastics comprise separating the analyte residue with size-exclusion chromatography and measuring a mass spectrum. As described above, using size-exclusion chromatography provides separation of the extracted monomers and polymers based on size, where a polymer can be a multiple of the size detected for the monomer. Using mass spectrometry provides an accurate measurement of the mass of each separated monomer and polymer, where again the polymer can be a multiple of the mass detected for the monomer. The mass measured is a unique qualifier of the monomer that makes up the microplastic polymer. As such, the present disclosure improves the identification of the microplastics from the biological sample based on both size and weight, providing two factors to ultimately identify the microplastics in biological samples. The size-exclusion chromatography method is not particularly limited and any size-exclusion chromatography instruction as known in the art can be used. In some embodiments, the size-exclusion chromatography is a reverse phase chromatography. In some embodiments, the size-exclusion chromatography is conducted with a reverse phase column that is at least 5 mm in diameter (e.g., at least 3 mm or 2 mm in diameter). In some embodiments, the size-exclusion chromatography uses a mobile phase comprising at least one of water, methanol, acetonitrile, and formic acid. In some embodiments, the size-exclusion chromatography uses a mobile phase comprising water, methanol, and formic acid. For example, in some embodiments, the mobile phase comprises no more than 99 vol % water, no more than 95 vol % methanol, and no more than 5 vol % formic acid. In some embodiments as described herein, the mobile phase comprises water in a range of 4 to 99 vol %, methanol in a range of 0 to 95 vol %, and formic acid in a range of 0.1 to 5 vol %. In various embodiments as described herein, the mobile phase comprises water, methanol, and formic acid in a volume ratio of at least 30:70:0.01, or at least 20:80:0.1, or at least 10:90:0.1. For example, in various embodiments, the mobile phase comprises water, methanol, and formic acid in a volume ratio in the range of 30:70:0.01 to 10:90:0.01. In some embodiments, the size-exclusion chromatography uses a mobile phase comprising water, acetonitrile and formic acid. For example, in some embodiments, the mobile phase comprises no more than 99 vol % water, no more than 95 vol % acetonitrile, and no more than 5 vol % formic acid. In some embodiments as described herein, the mobile phase comprises water in a range of 4 to 99 vol %, acetonitrile in a range of 0 to 95 vol %, and formic acid in a range of 0.1 to 5 vol %. In some embodiments, the mobile phase comprises water, acetonitrile, and formic acid in a volume ratio of at least 30:70:0.1, or at least 20:80:0.1, or at least 10:90:0.1. For example, in various embodiments, the mobile phase comprises water, acetonitrile, and formic acid in a volume ratio in the range of 30:70:0.01 to 10:90:0.01. In some embodiment as described herein, the mobile phase comprises water, acetonitrile, and formic acid in a volume ratio of at least 29:70:1, or at least 19:80:1, or at least 9:90:1. For example, in various embodiments as described herein, the mobile phase comprises water, acetonitrile, and formic acid in a volume ration in the range of 29:70:1 to 9:90:1. As described above, the mass measured is a unique qualifier of the monomer that makes up the microplastic polymer. The mass spectrometry method and instruments used can be any instrumentation as known in the art and is not particularly limited. For example, the ion source may be selected from an electrospray ionization source, an atmospheric pressure chemical ionization source, or an atmosphere pressure photo-ionization source and the mass analyzer may be selected from quadrupole analyzers, time-of-flight analyzers, ion trap analyzers, and hybrid analyzers. In particular examples of the disclosure, an electrospray ionization source and quadrupole mass analyzer is used. In some embodiment, the size-exclusion chromatography and mass spectrometry instrumentation are integrated together.

As described above, the depolymerization solutions disrupt the covalent bonds within polymer chains to provide polymers with a distribution of sizes. These can include monomers, dimers, trimers, and higher order polymers. As such, the mass spectrum can be acquired over any suitable range of m/z to measure the monomers and distribution of polymers. In various embodiments, the mass spectrum is acquired over the range or 1 to 500 m/z, or 10 to 500 m/z or 30 to 500 m/z, or 1 to 400 m/z, or 10 to 400 m/z, or 30 to 400 m/z, or 1 to 300 m/z, or 10 to 300 m/z, or 30 to 300 m/z. As would be understood by the person of ordinary skill in the art, the m/z does not necessarily directly correspond to the mass of the monomers and/or polymers measured as the charge on the molecule may vary (e.g., be greater than 1).

In various embodiments of the disclosure as described herein, the monomers have a molecular weight of at least 20 g/mol, or 30 g/mol, or 40 g/mol, or 50 g/mol. In various embodiments, the monomers have a molecular weight in the range of 20 to 200 g/mol, or 30 to 200 g/mol, or 40 to 200 g/mol, to 50 to 200 g/mol, or 20 to 180 g/mol, or 30 to 180 g/mol, or 40 to 180 g/mol, or 50 to 180 g/mol, or 20 to 160 g/mol, or 30 to 160 g/mol, or 40 to 160 g/mol, or 50 to 160 g/mol. As described above, the monomer of microplastic is not particularly limited and may originate from any plastic material (e.g., a thermoplastic or a thermosetting plastic). For example, in some embodiments of the present disclosure as described herein, the monomer is selected from methyl methacrylate, propylene, styrene, ethylene, or ethylene terephthalate.

As described above, polymer refers to a molecule having any number (i.e., n) of repeating monomers. Accordingly, in some embodiments as described herein, the polymer has a molecular weight that is at least 2 times (e.g., at least 3 times, at least 5 times, at least 10 times, or at least 50 times) of the molecular weight of the monomer. For example, in various embodiments as polymer has a molecular weight in the range of 2 to 300 times (e.g., in the range of 5 to 300 times, or 10 to 300 times, or 50 to 300 times, or 2 to 250 times, or 5 to 250 times, or 10 to 250 times, or 50 to 250 times) of the molecular weight of the monomer. In various embodiments as described herein, the polymer has a molecular weight in the range of 40 to 60,000 g/mol, or 60 to 60,000 g/mol, or 80 to 60,000 g/mol, to 100 to 60,000 g/mol, or 40 to 54,000 g/mol, or 60 to 54,000 g/mol, or 80 to 54,000 g/mol, or 100 to 54,000 g/mol, or 40 to 48,000 g/mol, or 60 to 48,000 g/mol, or 80 to 48,000 g/mol, or 100 to 48,000 g/mol. As described above, the polymer of microplastic is not particularly limited and may originate from any plastic material (e.g., a thermoplastic or a thermosetting plastic). For example, in some embodiments of the present disclosure as described herein, the polymers are selected from poly methyl methacrylate (PMMA), polypropylene (PP), polystyrene (PS), polyethylene (PE), polyethylene terephthalate (PET), or mixtures thereof.

In some embodiments as described herein, identifying the at least one microplastic comprises measuring a UV-Visible absorption of the analyte residue. The analyte residue may be eluted from the oversize and suspended in a carrier solution as described above before measuring the UV-Visible absorption. Measuring the UV-Visible absorption may be accomplished by any means known in the art and is not particularly limited. In various embodiments, the UV-Visible absorption is in the range of 200-500 nm, or 200-450 nm, or 200-400 nm.

In various embodiments, the monomers and/or polymers of the extracted microplastic have a UV-Visible absorption in the range of 200-500 nm, or 200-450 nm, or 200-400 nm. As described above, monomers and/or polymer of the microplastic are derived from larger plastic material. The type (e.g., polymer composition) of microplastic is not particularly limited and may originate from any plastic material (e.g., a thermoplastic or a thermosetting plastic). In some embodiments, the microplastics are thermoplastics. For example, in some embodiments of the present disclosure as described herein, the microplastics are selected from poly methyl methacrylate (PMMA), polypropylene (PP), polystyrene (PS), polyethylene (PE), polyethylene terephthalate (PET), or mixtures thereof.

Another aspect of the present disclosure provides a method for identifying at least one microplastic in a liquid blood sample. The method includes heating the liquid blood sample to provide a protein-containing sediment and a liquid phase disposed above the sediment; separating the liquid phase from the protein-containing sediment; evaporating the liquid phase to provide an analyte; treating the analyte with a depolymerization solution comprising an organic phase and an aqueous phase; extracting the analyte from the depolymerization solution in the organic phase of the depolymerization solution; filtering the organic phase to provide an analyte residue as the oversize; and identifying the at least one microplastic in the analyte residue with size-exclusion chromatography and mass spectrometry. This method may be accomplished by the heating, separating, evaporating, treating, extracting, filtering, and identification steps as described herein. Other examples are possible.

For example, in one example embodiment, microplastics may be identified using additional or alternative methods. In one example, microplastics may be identified using light detection and ranging (“LIDAR”) and/or other imaging techniques. For example, a computing device can be implemented as a controller, and the controller can be used to capture one or more images of a solution (e.g., blood, solvent, sample) containing one or more microplastics, as well as process a plurality of the captured images to generate and/or annotate a composite image of the plurality of images. For example, the controller and/or components operating therewith (e.g., a camera) may capture the images and perform a series of actions to further analyze the images. For example, the controller may stitch together the plurality of images of the sample solution that contains microplastics and remove any images that it determines do not contain microplastics. In example embodiments, the controller may determine which images in the plurality of images of the transferred sample of solution that contain particles by performing one or more of image processing steps on individual images in the plurality of images (e.g., pixel analysis, gradient analysis, light refraction analysis). In example embodiments, the controller may use one or more algorithms (e.g., machine learning and artificial intelligence algorithms) and/or detection protocols to automatically detect the microplastics in individual and/or composite images of the plurality of images, thereby determining a presence of microplastics in the images. Other examples are possible, including that the controller may generate a density, type, and/or count of the microplastics in the sample solution, and/or present annotated images of the microplastics identified in the images. In example embodiments, the controller may use on or more algorithms to identify the microplastics present in the sample by analyzing the light refraction indices. Other examples are possible.

Another aspect of the present disclosure provides a method of identifying at least one microplastic in a non-protein-containing liquid biological sample. As described above, when the biological sample does not include proteins, removal of unwanted substances from the sample is not necessary. As such, the method of identifying at least one microplastic in a non-protein-containing liquid biological sample comprises providing a non-protein-containing liquid biological sample; evaporating the non-protein-containing liquid biological sample to provide an analyte; treating the analyte with a depolymerization solution comprising an organic phase and an aqueous phase; extracting the analyte from the depolymerization solution in the organic phase of the depolymerization solution; filtering the organic phase to provide an analyte residue as the oversize; and identifying the at least one microplastic in the analyte residue. This method may be accomplished by the evaporating, treating, extracting, filtering, and identification steps as described herein.

The non-protein-containing liquid biological sample may be obtained from either a human or an animal. In some embodiments as described herein, the non-protein-containing liquid biological sample is obtained from a human. In some embodiments as described herein, the non-protein-containing liquid biological sample is obtained from an animal. The type of animal is not particularly limited. For example, the animal may be selected from a dog, a cat, a mouse or rat, or a bird. As described above, in some embodiments of the disclosure, the non-protein-containing liquid biological sample does not contain protein. For example, in some embodiments, the non-protein-containing liquid biological sample is a urine sample. Additionally, the liquid biological sample may be further treated before the heating step as described herein. For example, the liquid biological sample may be diluted with an aqueous solution. The aqueous solution is not particularly limited. For example, in some embodiments, the aqueous solution may be an aqueous alcohol solution (e.g., at least 10%, at least 25%, at least 50%, at least 75% aqueous alcohol solution). The alcohol may be selected from methanol, ethanol, or butanol.

The person of ordinary skill in the art will provide the materials and perform the methods described herein based on the general disclosure above.

### EXAMPLES

The Examples that follow are illustrative of specific embodiments of the processes of the disclosure and various uses thereof. They are set forth for explanatory purposes only, and are not to be taken as limiting the scope of the disclosure.

### Example 1. Measurement of Microplastics Standards

Four microplastic standards were measured using liquid-chromatography/mass spectrometry (LC/MS). Each microplastic standard include a polymer as described below in Table 1.

TABLE 1

| Sample | | | |
|---|---|---|---|
| No. | Identification | Source | Product No. |
| 1 | Polypropylene | Sigma Aldrich | 428116-1KG |
| 2 | Polyethylene | Sigma Aldrich | 429252-250G Lot# |
| | terephthalate | | WXBD6415V |
| 3 | Poly(methyl | Cospheric | PMPMS-1.2 1-75 um - |
| | methacrylate) | | 25 g |
| 4 | Clear | Cospheric | CPMS-0.96 10-90 um - |
| | polyethylene | | 10 g |

To prepare the standards, each polymer was combined with potassium hydroxide (KOH):1-butanol (1:10) solution at a concentration of 1M. The solution was then heated, under stirring until all the liquid evaporated (approximately 8 minutes). The remaining solids residue was suspended in methanol. The methanol was evaporated and the remaining solids were suspended again in an acetonitrile:H₂O (0.5:1) solution. The acetonitrile:H₂O solution including the polymer was diluted to an appropriate concentration for analysis by LC/MS.

A Prominence Liquid Chromatograph LC2-AD (from Shimadzu), equipped with a Nexera Autosample SIL-3AC (from Shimadzu), a Prominence Column Oven CTO-2AC (from Shimadzu), a Prominence Communication Bus Module CBM-20A (from Shimadzu), and a 5 μm EVO C18 100 Å LC column (from Kinetex) was used. A triple quadrupole linear ion trap mass spectrometer (QTRAP 4500, from AB Sciex) was used in tandem with the liquid chromatograph. The polymer standers were analyzed using LC/MS using a mobile phase comprising 1 vol % formic acid, 90 vol % acetonitrile, and 9 vol % H₂O. Table 2 describes the parameters used to detect the microplastic standards with the liquid chromatograph and mass spectrometer.

TABLE 2

| Liquid Chromatograph | | | Mass Spectrometry | | |
|---|---|---|---|---|---|
| Parameter | Value | | Parameters | Value | |
| Pump Flow | 0.200 | mL/min | Polarity | Positive and negative | |
| | | | | polarity | |
| Pump pressure | 1451 | PSI | Scan Rate | 10000 | D/s |
| maximum | | | | | |
| Pumping mode | Isocratic flow | | Start | 100 | Da |
| Needle stroke | 52 | mm | Stop | 2000 | Da |
| Control vial | 52 | mm | Cycle time | 5.335 | sec |
| needle stroke | | | | | |
| Sampling speed | 5.0 | uL/sec | Cycles | 217 | |
| Cooler | 15° | C. | Step size | 0.12 | Da |
| Temperature | | | | | |
| Measuring line | 100 | uL | Settling time | 50 | ms |
| purge volume | | | | | |
| Rinsing Speed | 35 | uL/sec | Curtain Gas | 35 | psi |
| Rinsing | 0.500 | mL | Collision | High | |
| Volume | | | Gas | | |
| Rinse mode | Before and after | | Ion Spray | +/−4500 | |
| | aspiration | | voltage | | |
| Rinsing | Methanol | | Temperature | 650° | C. |
| solution | | | | | |
| Rinse time | 2 | sec | Ion Source | 55 | psi |
| | | | Gas 1 | | |
| Purge time | 2 | min | Ion Source | 50 | psi |
| | | | Gas 2 | | |
| Over | 40° | C. | Declustering | 30 | V |
| temperature | | | Potential | | |
| Maximum oven | 85° | C. | Entrance | 10 | V |
| temperature | | | Potential | | |
| Run time | 20 | min | Collision | 10% | |
| | | | Energy | | |
| Injection | 15 | uL | — | — | |
| volume | | | | | |

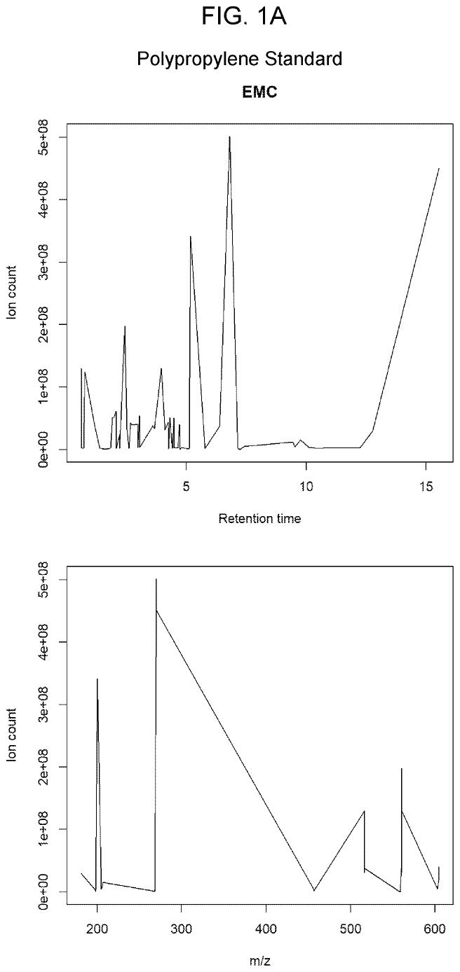

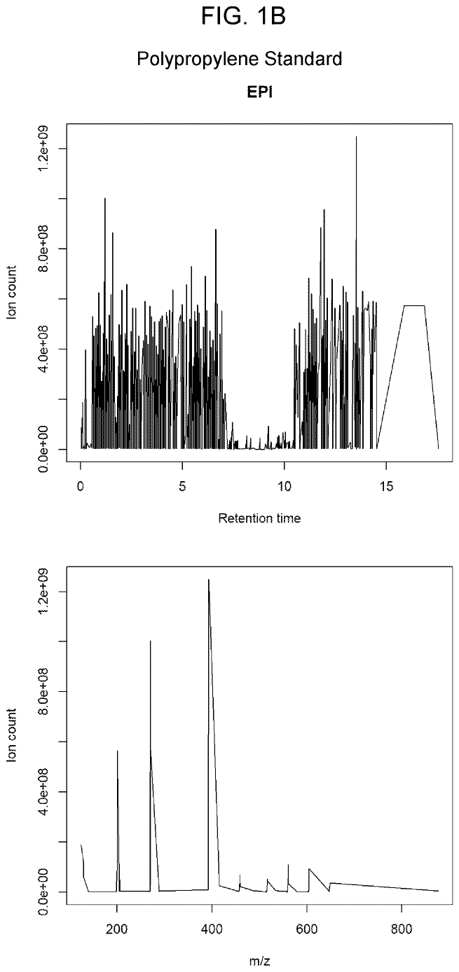

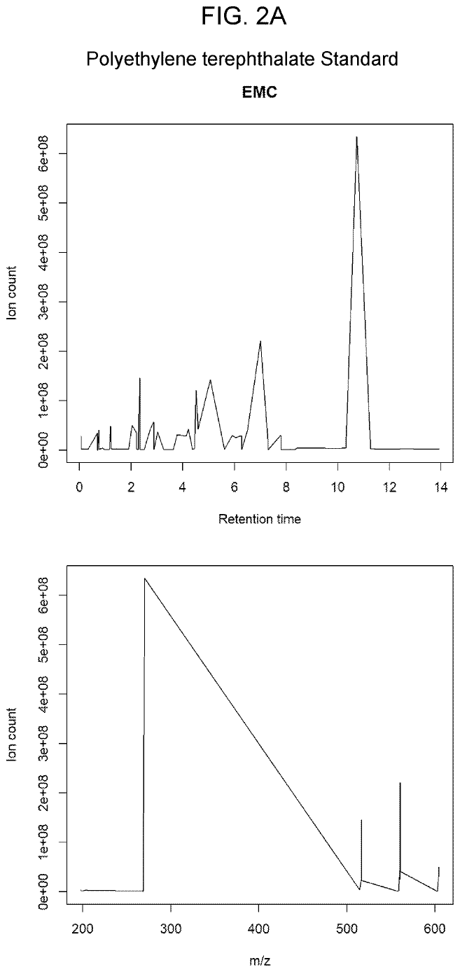

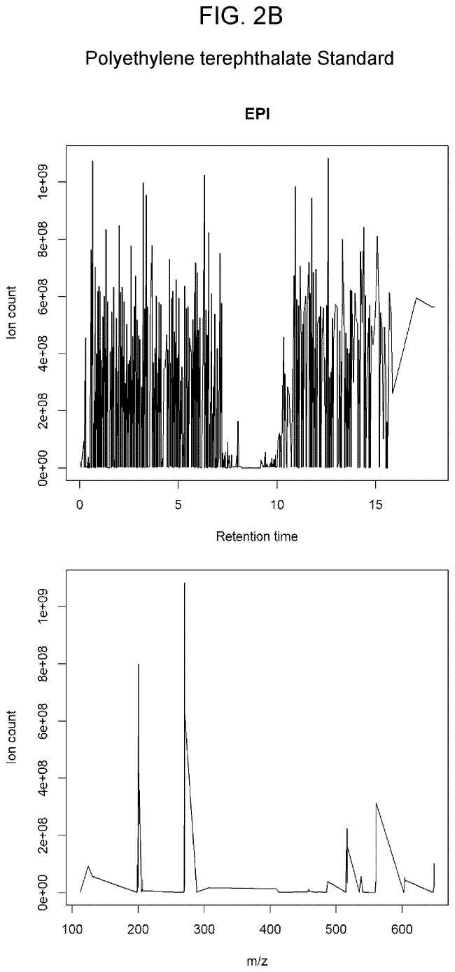

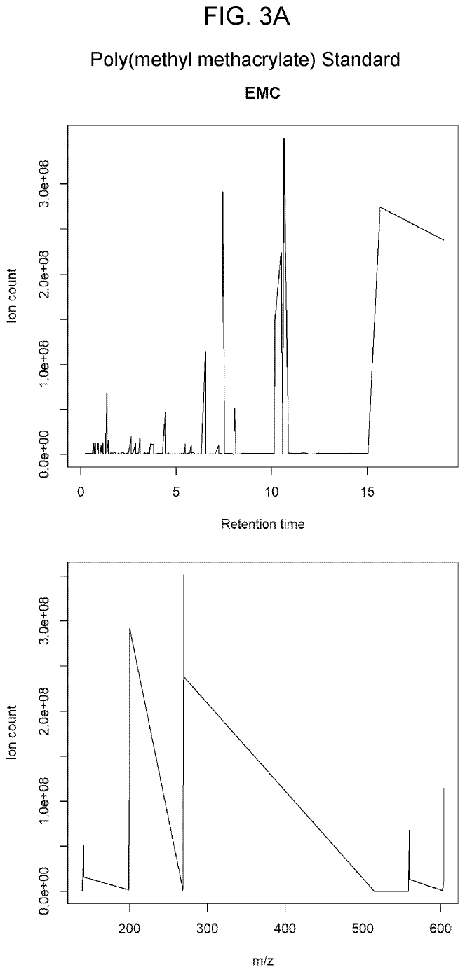

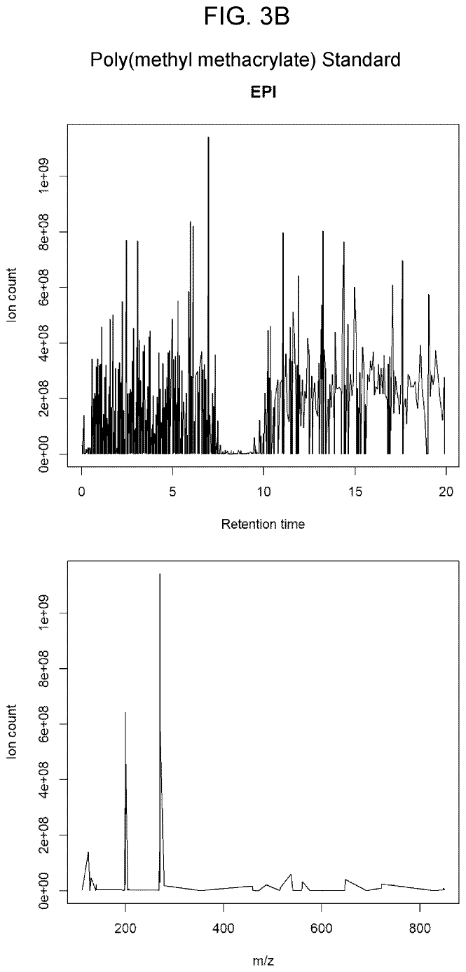

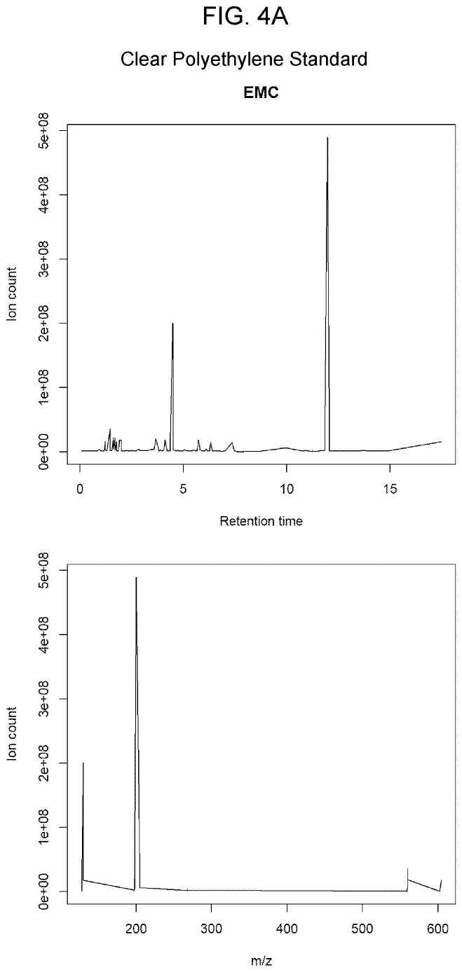

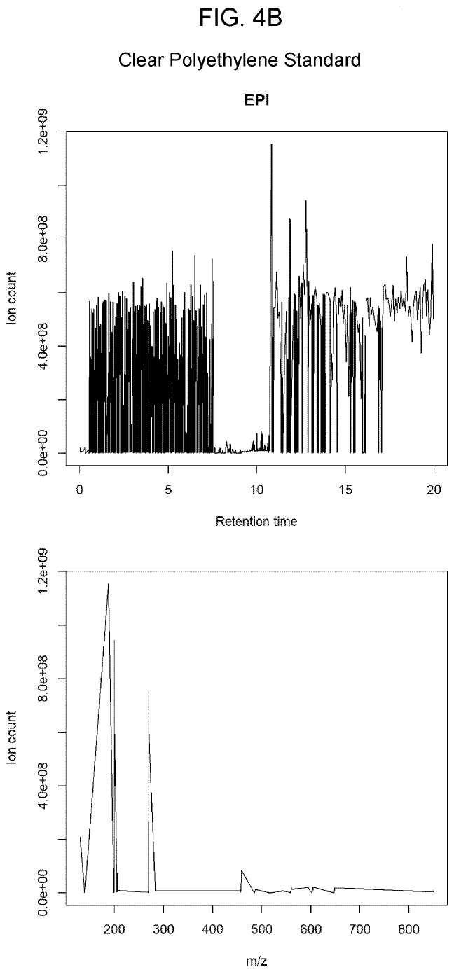

Two different ionization methods, Enhanced Multi-Charge (EMC) and Enhanced Product Ion (EPI), were used for the mass spectrometry of each microplastic standard. The retention time and mass spectrum for each standard (i.e., polypropylene shown in FIGS. 1A and 1B, polyethylene terephthalate shown in FIGS. 2A and 2B, poly(methyl methacrylate) shown in FIGS. 3A and 3B, and clear polyethylene shown in FIGS. 4A and 4B) are shown in FIGS. 1A-4B. Each microplastic standard provides a distinct spectral profile.

### Example 2. Measurement of Blood Samples

Two blood samples were measured by the LC/MS methods as described in Example 1 and Table 2. To prepare the blood samples, whole blood samples were diluted with red blood cell lysis buffer at a volume ration of 1:1 and 100 μg/mL Proteinase K was added to the diluted blood samples. This solution was added to a bead bath at 56° C. for 1 hour to break down proteins. The solution was then filtered over a glass fiber filter paper and the flow-through was discarded. The glass fiber filter paper was submerged in 10 mL of methanol (for 10 mL of blood) to allow for full elution of analytes from the filter paper. The liquid was collected and the methanol was evaporated. The analyte residue was suspended in an acetonitrile:H₂O (0.5:1) solution. This acetonitrile:H₂O solution, including the analytes, was diluted to an appropriate concentration for analysis by LC/MS.

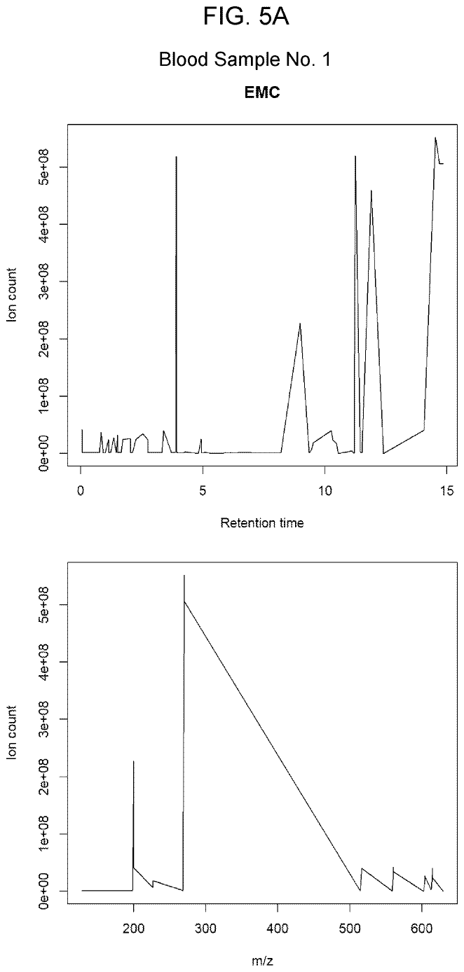

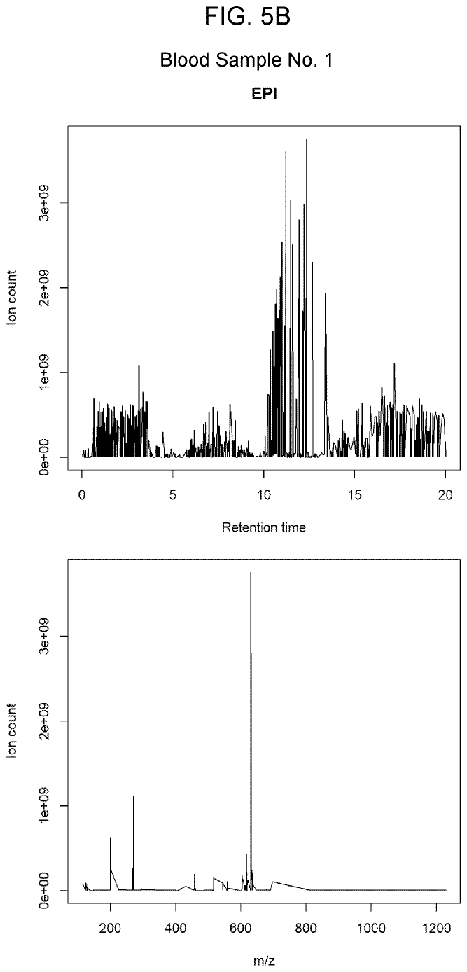

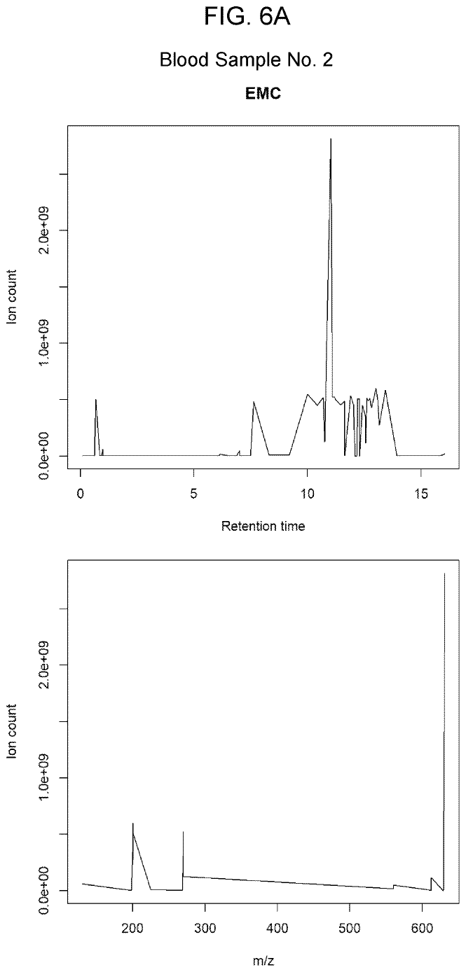

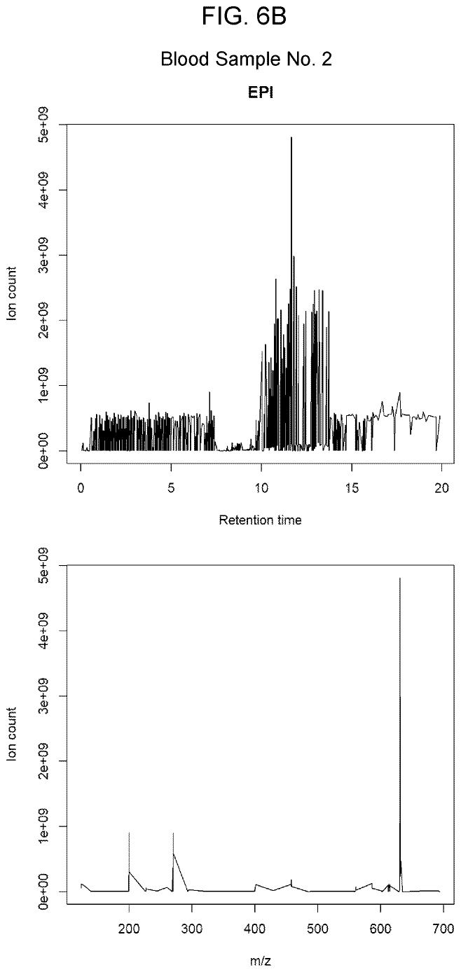

As with the microplastic standards, two different ionization methods, Enhanced Multi-Charge (EMC) and Enhanced Product Ion (EPI), were used for the mass spectrometry of each blood sample. The retention time and mass spectrum for each blood sample are shown in FIGS. 5A to 6B. Each blood sample provides a distinct spectral profile.

Additional aspects of the disclosure are provided by the following enumerated embodiments, which may be combined in any number and in any combination that is not logically or technically inconsistent.
Embodiment 1. A method for identifying at least one microplastic in a protein-containing liquid biological sample, wherein the method comprises:
   heating the protein-containing liquid biological sample to provide a protein-containing sediment and a liquid phase disposed above the sediment;
   separating the liquid phase from the protein-containing sediment;
   evaporating the liquid phase to provide an analyte;
   treating the analyte with a depolymerization solution comprising an organic phase and an aqueous phase;
   extracting the analyte from the depolymerization solution in the organic phase of the depolymerization solution;
   filtering the organic phase to provide an analyte residue as the oversize; and
   identifying the at least one microplastic in the analyte residue with size-exclusion liquid chromatography and mass spectrometry.
Embodiment 2. The method of embodiment 1, wherein the biological sample is obtained from a human or an animal.
Embodiment 3. The method of embodiment 1 or embodiment 2, wherein the biological sample is a blood sample.
Embodiment 4. The method of embodiment 3, wherein the blood sample is a liquid whole blood sample (e.g., comprises red blood cells, white blood cells, platelets, and plasma).
Embodiment 5. The method of any of embodiments 1-4, wherein heating the biological sample is conducted at a temperature in the range of 90° C. to 130° C. (e.g., in the range of 100° C. to 130° C., or 110° C. to 130° C., or 90 to 120° C., or 100° C. to 120° C., or 110° C. to 120° C.).
Embodiment 6. The method of any of embodiments 1-5, wherein heating the biological sample is conducted for a time in the range of 5 minutes to 1 hour (e.g., in the range of 5 to 50 minutes, or 5 to 40 minutes, or 10 minutes to 1 hour, or 10 to 50 minutes, or 10 to 40 minutes, or 20 minutes to 1 hour, or 20 to 50 minutes, or 20 to 40 minutes).
Embodiment 7. The method of any of embodiments 1-6, wherein heating the biological sample is conducted in a closed system, an open system, or a combination of both.
Embodiment 8. The method of any of embodiments 1-7, wherein the liquid phase comprises microplastics.
Embodiment 9. The method of embodiment 8, wherein the microplastics are selected from poly methyl methacrylate (PMMA), polypropylene (PP), polystyrene (PS), polyethylene (PE), polyethylene terephthalate (PET), or mixtures thereof.
Embodiment 10. The method of any of embodiments 1-9, wherein evaporating the liquid phase is conducted at a temperature in the range of 90° C. to 130° C. (e.g., in the range of 100° C. to 130° C., or 110° C. to 130° C., or 90 to 120° C., or 100° C. to 120° C., or 110° C. to 120° C.).
Embodiment 11. The method of any of embodiments 1-10, wherein evaporating the liquid phase is conducted for a time in the range of 5 minutes to 1 hour (e.g., in the range of 5 to 50 minutes, or 5 to 40 minutes, or 10 minutes to 1 hour, or 10 to 50 minutes, or 10 to 40 minutes, or 20 minutes to 1 hour, or 20 to 50 minutes, or 20 to 40 minutes).
Embodiment 12. The method of any of embodiments 1-11, wherein the depolymerization solution comprises an organic phase selected from methanol, ethanol, propanol, butanol, or pentanol.
Embodiment 13. The method of any of embodiments 1-12, wherein the depolymerization solution comprises an aqueous phase that is basic.
Embodiment 14. The method of any or embodiments 1-13, wherein the depolymerization solution comprises an organic phase and an aqueous phase in a volume ratio of at least 4:1 (e.g., at least 6:1, or at least 8:1, or at least 10:1).
Embodiment 15. The method of any of embodiments 1-14, wherein extracting the analyte from the depolymerization solution comprises heating the depolymerization solution, shaking the depolymerization solution, and collecting the organic phase.
Embodiment 16. The method of embodiment 15, wherein heating the depolymerization solution is conducted for a time in the range of 10 to 120 minutes (e.g., in the range of 10 to 100 minutes, or 10 to 80 minutes, or 10 to 60 minutes, or 20 to 120 minutes, or 20 to 100 minutes, or 20 to 80 minutes, or 20 to 60 minutes).
Embodiment 17. The method of embodiment 15 or embodiment 16 wherein heating the depolymerization solution is conducted at a temperature of in the range of 50-200° C. (e.g., in the range of 50-175° C., or 50-150° C., or 65-200° C., or 65-175° C., or 65-150° C., or 75-200° C., or 75-175° C., or 75-150° C., or 85-200° C., or 85-175° C., or 85-150° C., or 95-200° C., or 95-175° C., or 95-150° C.).
Embodiment 18. The method of any of embodiments 15-17, wherein collecting the organic phase comprises separating (e.g., by centrifugation) the organic phase from the aqueous phase of the depolymerization solution.
Embodiment 19. The method of embodiment 18, further comprising adjusting the pH of the organic phase to a pH in the range of 2 to 5 (e.g., 2 to 4, or 2 to 3).
Embodiment 20. The method of any of embodiments 1-19, wherein the organic phase comprises the analyte.
Embodiment 21. The method of any of embodiments 1-20, wherein filtering the organic phase comprises a filter of at least 200 nm (e.g., at least 300 nm, or 400 nm, or 500 nm, or 600 nm, or 700 nm).
Embodiment 22. The method of any of embodiments 1-20, wherein the filtering comprises a filter with a pore size in the range of 100 to 1000 nm (e.g., 100 to 800 nm, or 200 to 1000 nm, or 200 to 800 nm, or 500 to 1000 nm, or 500 to 800 nm).
Embodiment 23. The method of any of embodiments 1-22, wherein the analyte residue (i.e., oversize) is provided on the filter.
Embodiment 24. The method of any of embodiments 1-23, further comprises preparing the oversize for identification.
Embodiment 25. The method of embodiment 24, wherein preparing the oversize for identification comprises eluting the analyte reside from the filter with an organic solvent (e.g., methanol, ethanol, or butanol), evaporating the organic solvent, and suspending the oversize in a carrier solution.
Embodiment 26. The method of embodiment 25, wherein the carrier solution comprises acetonitrile and water (e.g., in a volume ratio of 1:1).
Embodiment 27. The method of any of embodiments 1-26, wherein identifying the at least one microplastics in the analyte residue comprises measuring the carrier solution comprising the eluted analyte residue.
Embodiment 28. The method of any of embodiments 1-27, wherein identifying the at least one microplastics comprises separating the analyte residue with size-exclusion chromatography and measuring a mass spectrum.
Embodiment 29. The method of embodiment 28, wherein the size-exclusion chromatography is a reverse phase chromatography.
Embodiment 30. The method of embodiments 28 or 29, wherein the size-exclusion chromatography is conducted with a reverse phase column that is at least 5 mm in diameter.
Embodiment 31. The method of any of embodiments 28-30, wherein the size exclusion chromatography uses a mobile phase comprising at least one of water, methanol, acetonitrile, and formic acid.
Embodiment 32. The method of any of embodiments 28-31, wherein the size-exclusion chromatography uses a mobile phase comprising water, methanol, and formic acid.
Embodiment 33. The method of embedment 32, wherein the mobile phase comprises no more than 99 vol % water, no more than 95 vol % methanol, and no more than 5 vol % formic acid.
Embodiment 34. The method of embodiment 32, wherein the mobile phase comprises water in a range of 4 to 99 vol %, methanol in a range of 0 to 95 vol %, and formic acid in a range of 0.1 to 5 vol %.
Embodiment 35. The method of embodiment 32, wherein the mobile phase comprises water, methanol, and formic acid in a volume ratio of at least 30:70:0.01 (e.g., at least 20:80:0.01, or at least 10:90:0.01).
Embodiment 36. The method of any of embodiments 28-31, wherein the size-exclusion chromatography uses a mobile phase comprising water, acetonitrile, and formic acid.
Embodiment 37. The method of embedment 36, wherein the mobile phase comprises no more than 99 vol % water, no more than 95 vol % acetonitrile, and no more than 5 vol % formic acid.
Embodiment 38. The method of embodiment 36, wherein the mobile phase comprises water in a range of 4 to 99 vol %, acetonitrile in a range of 0 to 95 vol %, and formic acid in a range of 0.1 to 5 vol %.
Embodiment 39. The method of embodiment 36, wherein the mobile phase comprises water, acetonitrile, and formic acid in a volume ratio of at least 30:70:0.01 (e.g., at least 20:80:0.01, or at least 10:90:0.01).
Embodiment 40. The method of any of embodiments 28-39, wherein the mass spectrum is acquired over the range of 1 to 500 m/z (e.g., over the range of 10 to 500 m/z or 30 to 500 m/z, or 1 to 400 m/z, or 10 to 400 m/z, or 30 to 400 m/z, or 1 to 300 m/z, or 10 to 300 m/z, or 30 to 300 m/z).
Embodiment 41. The method of any of embodiments 1-40, wherein identifying the at least one microplastic comprises measuring a UV-Visible absorption.
Embodiment 42. The method of embodiment 41, wherein the UV-Visible absorption is in the range of 200-500 nm.
Embodiment 43. The method of any of embodiments 1-42, wherein a monomer of the microplastic has a molecular weight in the range of 20 to 200 g/mol.
Embodiment 44. The method of any of embodiments 1-43, wherein a polymer of the microplastic have a molecular weight in the range of 40 to 60.00 g/mol.
Embodiment 45. The method of any of embodiments 1-44, wherein a monomer and/or polymer of the microplastics has a UV-Visible absorption in the range of 200-500 nm.
Embodiment 46. The method of any of embodiments 1-45, wherein a monomer of the microplastic is selected from methyl methacrylate, propylene, styrene, ethylene, or ethylene terephthalate.
Embodiment 47. The method of any of embodiments 1-46, wherein a polymer of the microplastic is selected from poly methyl methacrylate (PMMA), polypropylene (PP), polystyrene (PS), polyethylene (PE), or polyethylene terephthalate (PET).
Embodiment 48. A method for identifying at least one microplastic in a liquid blood sample, wherein the method comprises:
   heating the liquid blood sample to provide a protein-containing sediment and a liquid phase disposed above the sediment,
   separating the liquid phase from the protein-containing sediment;
   evaporating the liquid phase to provide an analyte;
   treating the analyte with a depolymerization solution comprising an organic phase and an aqueous phase;
   extracting the analyte from the depolymerization solution in the organic phase of the depolymerization solution;
   filtering the organic phase to provide an analyte residue as the oversize; and
   identifying the at least one microplastic in the analyte residue with size-exclusion chromatography and mass spectrometry.
Embodiment 49. A method for identifying at least one microplastic in a non-protein-containing liquid biological sample, wherein the method comprises:
   providing a non-protein-containing liquid biological sample;
   evaporating the non-protein-containing liquid biologic sample to provide an analyte;
   treating the analyte with a depolymerization solution comprising an organic phase and an aqueous phase;
   extracting the analyte from the depolymerization solution in the organic phase of the depolymerization solution;
   filtering the organic phase to provide an analyte residue as the oversize; and
   identifying the at least one microplastic in the analyte residue.
Embodiment 50. The method of embodiment 49, wherein the biological sample is a urine sample.
Embodiment 51. The method of embodiment 49 or embodiment 50, wherein the microplastics are selected from poly methyl methacrylate (PMMA), polypropylene (PP), polystyrene (PS), polyethylene (PE), polyethylene terephthalate (PET), or mixtures thereof.
Embodiment 52. The method of any of embodiments 49-51, wherein the depolymerization solution comprises an organic phase (e.g. alcohol, methanol, ethanol, or butanol) and an aqueous phase (e.g., basic, sodium hydroxide or potassium hydroxide).
Embodiment 53. The method of embodiment 49-52, wherein the depolymerization solution comprises an organic phase and an aqueous phase in a volume ratio of at least 4:1.
Embodiment 54. The method of embodiment any of embodiments 49-53, wherein extracting the analyte from the depolymerization solution comprises heating the depolymerization solution, shaking the depolymerization solution, and collecting the organic phase.
Embodiment 55. The method of embodiment 54, wherein heating the depolymerization solution is conducted for a time in the range of 10 to 120 minutes (e.g., in the range of 10 to 100 minutes, or 10 to 80 minutes, or 10 to 60 minutes, or 20 to 120 minutes, or 20 to 100 minutes, or 20 to 80 minutes, or 20 to 60 minutes).
Embodiment 56. The method of embodiment 54 or embodiment 55 wherein heating the depolymerization solution is conducted at a temperature in the range of 50-200° C.
Embodiment 57. The method of any of embodiments 54-56, wherein collecting the organic phase comprises separating (e.g., by centrifugation) the organic phase from the aqueous phase of the depolymerization solution.
Embodiment 58. The method of embodiment 57, further comprising adjusting the pH of the organic phase to a pH in the range of 2 to 5 (e.g., 2 to 4, or 2 to 3).
Embodiment 59. The method of any of embodiments 49-58, wherein the organic phase comprises the analyte.
Embodiment 60. The method of any of embodiments 49-59, wherein filtering the organic phase comprises a filter of at least 200 nm (e.g., at least 300 nm, or 400 nm, or 500 nm, or 600 nm, or 700 nm).
Embodiment 61. The method of any of embodiments 49-59, wherein the filtering comprises a filter with a pore size in the range of 100 to 1000 nm (e.g., 100 to 800 nm, or 200 to 1000 nm, or 200 to 800 nm, or 500 to 1000 nm, or 500 to 800 nm).
Embodiment 62. The method of any of embodiments 49-61, wherein the analyte residue (i.e., oversize) is provided on the filter.
Embodiment 63. The method of any of embodiments 49-62, further comprises preparing the analyte residue for analysis.
Embodiment 64. The method of embodiment 63, wherein preparing the analyte residue for identification comprises eluting the analyte residue from the filter with an organic solvent (e.g., methanol, ethanol, or butanol), evaporating the organic solvent, and suspending the oversize in a carrier solution.
Embodiment 65. The method of embodiment 64, wherein the carrier solution comprises acetonitrile and water (e.g., in a volume ratio of 1:1).
Embodiment 66. The method of any of embodiments 49-65, wherein identifying at least one microplastics in the analyte residue comprises measuring the carrier solution comprising the eluted analyte residue.
Embodiment 67. The method of any of embodiments 49-66, wherein identifying at least one microplastics comprises separating the analyte residue with size-exclusion chromatography and measuring a mass spectrum.
Embodiment 68. The method of embodiment 67, wherein the size-exclusion chromatography is a reverse phase chromatography.
Embodiment 69. The method of embodiments 67 or 68, wherein the size-exclusion chromatography is conducted at a reverse phase column that is at least 5 mm in diameter.
Embodiment 70. The method of any of embodiments 67-69, wherein the size exclusion chromatography uses a mobile phase comprising at least one of water, methanol, acetonitrile, and formic acid.
Embodiment 71. The method of any of embodiments 67-69, wherein the size-exclusion chromatography uses a mobile phase comprising water, methanol, and formic acid.
Embodiment 72. The method of embodiment 71, wherein the mobile phase comprises no more than 99 vol % water, no more than 95 vol % methanol, and no more than 5 vol % formic acid.
Embodiment 73. The method of embodiment 71, wherein the mobile phase comprises water in a range of 4 to 99 vol %, methanol in a range of 0 to 95 vol %, and formic acid in a range of 0.1 to 5 vol %.
Embodiment 74. The method of embodiment 71, wherein the mobile phase comprises water, methanol, and formic acid in a volume ratio of at least 30:70:0.01 (e.g., at least 20:80:0.01, or at least 10:90:0.01).
Embodiment 75. The method of any of embodiments 67-69, wherein the size-exclusion chromatography uses a mobile phase comprising water, acetonitrile, and formic acid.
Embodiment 76. The method of embodiment 75, wherein the mobile phase comprises no more than 99 vol % water, no more than 95 vol % acetonitrile, and no more than 5 vol % formic acid.
Embodiment 77. The method of embodiment 75, wherein the mobile phase comprises water in a range of 4 to 99 vol %, acetonitrile in a range of 0 to 95 vol %, and formic acid in a range of 0.1 to 5 vol %.
Embodiment 78. The method of embodiment 75, wherein the mobile phase comprises water, acetonitrile, and formic acid in a volume ratio of at least 30:70:0.01 (e.g., at least 20:80:0.01, or at least 10:90:0.01).
Embodiment 79. The method of any of embodiments 49-78, wherein identifying at least one microplastics comprises measuring a UV-Visible absorption.
Embodiment 80. The method of embodiment 79, wherein the UV-Visible absorption is in the range of 200-500 nm.
Embodiment 81. The method of any of embodiments 49-80, wherein a monomer of the microplastic has a molecular weight in the range of 20 to 200 g/mol.
Embodiment 82. The method of any of embodiments 49-81, wherein a polymer of the microplastic have a molecular weight in the range of 40 to 60.00 g/mol.
Embodiment 83. The method of any of embodiments 49-82, wherein a monomer and/or polymer of the microplastics has a UV-Visible absorption in the range of 200-500 nm.
Embodiment 84. The method of any of embodiments 49-83, wherein a monomer of the microplastic is selected from methyl methacrylate, propylene, styrene, ethylene, or ethylene terephthalate.
Embodiment 85. The method of any of embodiments 49-83, wherein a polymer of the microplastic is selected from poly methyl methacrylate (PMMA), polypropylene (PP), polystyrene (PS), polyethylene (PE), or polyethylene terephthalate (PET).

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this disclosure are described herein, including the best mode known to the inventors for carrying out the disclosure. Variations on these described embodiments will become apparent to those of ordinary skill in the art upon reading the foregoing description and skilled artisans will be able to employ such variations as appropriate. The embodiments described in the present disclosure are merely for illustrative purposes and may be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A method for identifying at least one microplastic in a protein-containing liquid biological sample, wherein the method comprises:
heating the protein-containing liquid biological sample to provide a protein-containing sediment and a liquid phase disposed above the sediment;
separating the liquid phase from the protein-containing sediment;
evaporating the liquid phase to provide an analyte;
treating the analyte with a depolymerization solution comprising an organic phase and an aqueous phase;
extracting the analyte from the depolymerization solution in the organic phase of the depolymerization solution;
filtering the organic phase to provide an analyte residue as the oversize; and
identifying the at least one microplastic in the analyte residue with size-exclusion liquid chromatography and mass spectrometry.

2. (canceled)

3. The method of claim 1, wherein the biological sample is a blood sample.

4. The method of claim 3, wherein the blood sample is a liquid whole blood sample.

5. The method of claim 1, wherein heating the biological sample is conducted at a temperature in the range of 90° C. to 130° C.

6. The method of any of claims 1-5, wherein heating the biological sample is conducted for a time in the range of 5 minutes to 1 hour.

7. The method of claim 1, wherein the liquid phase comprises microplastics.

8. The method of claim 1, wherein the depolymerization solution comprises an organic phase selected from methanol, ethanol, propanol, butanol, or pentanol.

9. The method of claim 1, wherein the depolymerization solution comprises an aqueous phase that is basic.

10. The method of claim 1, wherein the depolymerization solution comprises an organic phase and an aqueous phase in a volume ratio of at least 4:1.

11. The method of claim 1, wherein extracting the analyte from the depolymerization solution comprises heating the depolymerization solution, shaking the depolymerization solution, and collecting the organic phase.

12. The method of claim 1, wherein the organic phase comprises the analyte.

13. The method of claim 1, wherein filtering the organic phase comprises a filter of at least 200 nm.

14. (canceled)

15. The method of claim 1, further comprises preparing the oversize for identification, wherein preparing the oversize for identification comprises eluting the analyte reside from the filter with an organic solvent, evaporating the organic solvent, and suspending the oversize in a carrier solution.

16. (canceled)

17. The method of claim 1, wherein identifying the at least one microplastics in the analyte residue comprises measuring the carrier solution comprising the eluted analyte residue.

18. The method of claim 1, wherein identifying the at least one microplastics comprises separating the analyte residue with size-exclusion chromatography and measuring a mass spectrum.

19. The method of claim 1, wherein a monomer of the microplastic has a molecular weight in the range of 20 to 200 g/mol, or a polymer of the microplastic have a molecular weight in the range of 40 to 60,00 q/mol.

20. (canceled)

21. The method of claim 1, wherein a monomer of the microplastic is selected from methyl methacrylate, propylene, styrene, ethylene, or ethylene terephthalate.

22. The method of claim 1, wherein a polymer of the microplastic is selected from poly methyl methacrylate (PMMA), polypropylene (PP), polystyrene (PS), polyethylene (PE), or polyethylene terephthalate (PET).

23. A method for identifying at least one microplastic in a non-protein-containing liquid biological sample, wherein the method comprises:
providing the non-protein-containing liquid biological sample;
evaporating the non-protein-containing liquid biologic sample to provide an analyte;
treating the analyte with a depolymerization solution comprising an organic phase and an aqueous phase;
extracting the analyte from the depolymerization solution in the organic phase of the depolymerization solution;
filtering the organic phase to provide an analyte residue as the oversize; and
identifying the at least one microplastic in the analyte residue.

24. The method of claim 23, wherein the biological sample is a urine sample.
